(19) United States
(12) Patent Application Publication
(10) Pub. No.: US 2025/0299837 A1
(43) Pub. Date: Sep. 25, 2025

(54) MEDICAL DEVICE TRAINING PLATFORM USING REINFORCED LEARNING BASED ON HISTORICAL PRACTICE

(71) Applicant: KONINKLIJKE PHILIPS N.V., EINDHOVEN (NL)

(72) Inventors: Paul Anthony SHRUBSOLE, ARNHEM (NL); Milosh STOLIKJ, EINDHOVEN (NL); Douglas MCKNIGHT, CHARDON, OH (US)

(21) Appl. No.: 19/229,023

(22) Filed: Jun. 5, 2025

Related U.S. Application Data
(63) Continuation-in-part of application No. 18/218,136, filed on Jul. 5, 2023.
(60) Provisional application No. 63/359,986, filed on Jul. 11, 2022.

(51) Int. Cl. G16H 70/20; G16H 15/00

(52) U.S. Cl. CPC G16H 70/20; G16H 15/00

(57) ABSTRACT

At least one database stores clinical activity data indicative of clinical activities of medical professionals or maintenance activity by medical equipment servicing personnel. Consumption of educational content units related to one or more medical devices by a medical professional (or maintenance thereof by a servicing person) is tracked. Future clinical (or maintenance) activities to be performed by the medical professional (or servicing person) is predicted based on the clinical (or maintenance) activity data. One or more metrics are calculated related to the medical professional's (or servicing person's) knowledge and/or experience for a future time. The metrics may include a knowledge metric based on the tracked consumption of educational content units, and/or an experience metric based on the clinical (or maintenance) activity data and the predicted future clinical (or servicing) activities. One or more refreshment educational content units are recommended based on the one or more metrics.

### CROSS REFERENCE TO RELATED APPLICATIONS

The present application is a continuation-in-part of U.S. patent application Ser. No. 18/218,136, filed Jul. 5, 2023 which claims the benefit of U.S. Provisional Patent Application No. 63/359,986, filed Jul. 11, 2022. These applications are hereby incorporated by reference herein.

### FIELD

The following relates generally to the medical arts, medical education arts, and medical device operations educational content tracking arts, especially as directed to medical imaging devices.

### BACKGROUND

Complex medical devices offer great flexibility in how they can be used to diagnose, monitor, or treat patients. The performance of the medical device may depend strongly on how the operator uses the device, e.g. setting a non-optimal configuration may provide suboptimal results whereas using a more optimal configuration may provide better results. Moreover, medical devices that are connected to the Internet or another electronic network may receive software or firmware upgrades over the network that provide new features or enhance existing features; however, these may be useless if the operator is not trained to effectively use the new or enhanced features. Thus, there is substantial benefit to offering education and support to get the best results from the medical devices according to the clinical needs of patients and according to the specializations, way of working of the staff, and the type of hospital or clinical practice.

Health care professionals need to be prepared for unfamiliar situations they might be presented with during their daily practice. To do this, they need to learn how to conduct specific procedures, workflows, protocols, or practices for certain clinical cases/patient-situations that are presented to them. Since such highly specific situations or cases occur infrequently, the training and education that they receive is not as effective as it could be because the specific knowledge that was gained about how to deal with such cases will fade over time.

Staying up to date with best practices whilst preventing knowledge erosion over time is a common phenomenon in many skill-based and knowledge-based industries. This problem is exacerbated in the healthcare sector, given the high workload and stress of clinical staff. Moreover, it is difficult for health workers to have sufficient repertoire of knowledge at their disposal to deal with all different patient situations, medical device configurations or medical procedures.

Current education and training solutions may require a dedicated training system particularly when dealing with sensitive patient or personal information. Furthermore, education and training solutions try to address the above problem of knowledge decay by offering refresher courses to keep health workers up to date at regular (monthly or yearly) intervals. However, such solutions are not sensitive to the user's experience in the field and are not able to consider individual cases that learners will be confronted with in the future. Moreover, reference materials can readily go out of date, to be superseded by best practices from medical governance organizations etc. in which the context information about the learner's prior experience is not known.

The following discloses certain improvements to overcome these problems and others.

### SUMMARY

In one aspect, a non-transitory computer readable medium stores at least one database storing clinical activity data indicative of clinical activities of a plurality of medical professionals, and instructions readable and executable by at least one electronic processor to perform an educational refreshment method. That method includes: tracking consumption of educational content units related to one or more medical devices by a medical professional; predicting future clinical activities to be performed by the medical professional based on the clinical activity data; calculating one or more metrics related to the medical professional's knowledge and/or experience for a future time, the metrics including a knowledge metric calculated based on the tracked consumption of educational content units and/or an experience metric calculated based on the clinical activity data and the predicted future clinical activities; and recommending one or more refreshment educational content units based on the one or more metrics meeting an educational content refreshment criterion.

In another aspect, a non-transitory computer readable medium stores at least one database storing clinical activity data indicative of clinical activities of a plurality of medical professionals, and instructions readable and executable by at least one electronic processor to perform an educational refreshment method. That method includes: tracking consumption of educational content units related to one or more medical devices by a medical professional; predicting future clinical activities to be performed by the medical professional based on the clinical activity data; calculating a knowledge metric related to the medical professional's knowledge and/or experience for a future time; and recommending one or more refreshment educational content units based on the knowledge metric meeting an educational content refreshment criterion.

In another aspect, an educational refreshment method is disclosed for recommending educational content for medical equipment servicing personnel. The method includes: tracking consumption of educational content units related to maintenance of one or more medical devices by a medical equipment servicing person; predicting future medical equipment maintenance activities to be performed by the medical equipment servicing person based on medical equipment servicing activity data indicative of medical equipment servicing activities of a plurality of medical equipment servicing personnel; calculating an experience metric based on the medical equipment servicing activity data and the predicted future medical equipment maintenance activities; and recommending one or more refreshment educational content units based on the experience metric meeting an educational content refreshment criterion. In some embodiments, the calculation of the experience metric includes calculating an erosion of experience over a time interval ending at the future time over which the erosion of experience of the medical equipment servicing person occurs. In some embodiments, the predicting of future clinical activities performed by the medical equipment servicing person is further based on one or more of a schedule of the medical equipment servicing person, statistics related to performance of maintenance activity by the medical equipment servicing person, and data related to maintenance of the one or more medical devices. In some embodiments, the calculated experience metric relates to the medical equipment servicing person's experience relating to future medical equipment servicing activities predicted to be performed by the medical equipment servicing person. In some embodiments, the method further includes retrieving one or more refreshment educational content units from a database, and presenting the retrieved one or more refreshment educational content units to the medical equipment servicing person at a time prior to the future time.

One advantage resides in providing healthcare professionals with up-to-date training materials, while accounting for erosion of knowledge and experience over time.

Another advantage resides in pushing educational content to healthcare professionals in a timely and personalized fashion.

Another advantage resides in providing educational content to healthcare professionals tailored to the healthcare professional's pre-existing knowledge and experience, while again taking into account erosion of such knowledge/experience.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for monitoring educational content units in accordance with the present disclosure.

FIG. 2 shows exemplary flow chart operations of the system of FIG. 1.

FIG. 3 shows another embodiment of the system of FIG. 1.

### DETAILED DESCRIPTION

As used herein, a “medical procedure” refers to a medical workflow utilizing one or more medical devices to achieve a clinical purpose such as diagnosing a medical condition, providing a clinical therapy or treatment, screening for a specific illness, and/or so forth. Some examples of a medical procedure include, by way of non-limiting illustrative example: a medical imaging procedure utilizing a magnetic resonance imaging (MRI) scanner; an image guided therapy (IGT) procedure such as a catheterization procedure, biopsy procedure, or the like performed using an interventional instrument (e.g. catheter, biopsy needle, etc.), a medical imaging device providing real-time images for guidance during the IGT procedure, and possibly one or more patient monitoring devices; an oncology radiation therapy procedure employing a linear accelerator (LINAC) and associated control computer; a mammography procedure for breast cancer screening; and so forth. It is also contemplated for the medical procedure to be related to a task other than imaging or radiation therapy, such as setting up a patient on a mechanical ventilator.

The following relates to a system for proactively recommending and pushing educational refresher content. The educational content may be delivered via various pathways depending on the embodiment. A vendor of a medical device for a medical facility may provide an in-house online learning center, and/or outside online educational services may be used, as some illustrative examples. Such services can make it possible to collect data about the wide range of experiences a person has within online and offline training activities. However, a deficiency in these existing programs relates to refresher content. Existing educational systems are typically not effective at determining what types of refresher content would be beneficial for a given individual (e.g., imaging technician or radiologist).

The disclosed refresher content system defines areas where refreshment might be useful as “activities,” and leverages existing databases to mine information about consumed educational content and experiential content for an individual. For example, as relates to administration of contrast agent C, refreshment could be by way of consuming training material on this activity (educational content) or by way of actually performing imaging procedures that entail administration of contrast agent C (experiential content).

The following further recognizes that an individual's capability for a given activity will erode over time in the absence of refreshment. A formulation for tracking this in a quantitative manner is provided. The erosion of education for a given activity a is given by illustrative metrics kₐ(t+δ) and eₐ(t+δ), respectively, where δ is a time interval over which the erosion occurs, kₐ refers to knowledge erosion, and eₐ refers to experience erosion. These are computed in a predictive manner in some embodiments, based on upcoming training and work schedules.

The following further contemplates that educational content units (e.g. courses) may be broken down into small easily consumable segments such as short quizzes or course summaries, with these segments being tagged as to quantitative activity content. Thus, when it is predicted that an individual's training in an activity a will erode to the point of creating a knowledge gap this can be matched to educational content (real time or on-demand) that is then recommended to the individual. This can be done in a predictive fashion, so that for example the system may predict refreshment content for an activity may be optimally consumed two months from today.

The system in some embodiments recommends such refresher content, while in other embodiments the refresher content can be actively pushed to the individual, e.g. via a personalized feed of the online learning center.

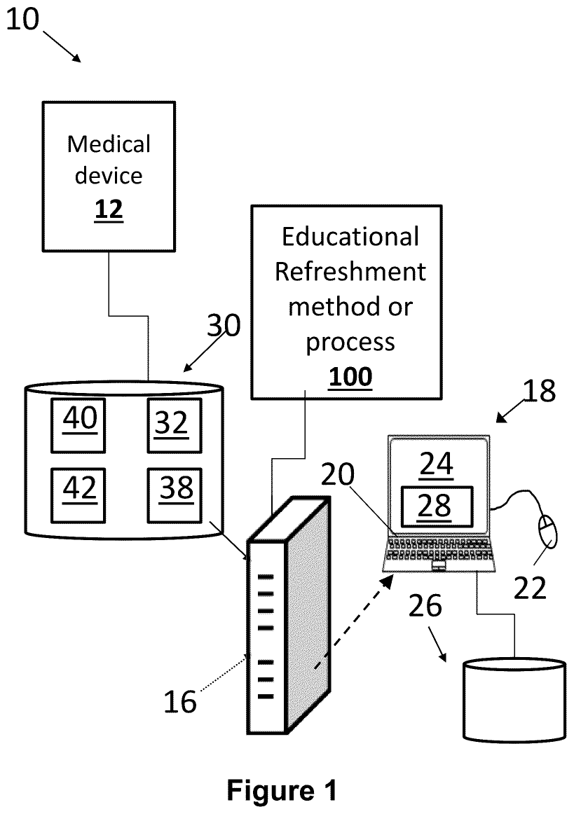

With reference to FIG. 1, an illustrative educational content monitoring system or apparatus 10 for monitoring educational content for a medical procedure employing one or more medical devices 12 (e.g., a medical imaging device 12; or a radiation therapy device; or a combination of the medical imaging device 12 and a biopsy needle, catheter, or other interventional instrument used cooperatively to perform an image guided therapy (IGT) procedure; or so forth). By way of some non-limiting illustrative examples, the medical imaging device 12 may be an interventional X-ray (IXR) or other interventional radiology (IR) system (used in combination with at least one interventional instrument in an IGT procedure), a magnetic resonance imaging (MRI) scanner, a computed tomography (CT) scanner, a positron emission tomography (PET) scanner, a gamma camera for performing single photon emission computed tomography (SPECT), or so forth. As shown in FIG. 1, the educational content generation system 10 includes, or is accessible by, a server computer 16 typically disposed remotely from the medical device(s) 12 used in the medical procedure for which content is to be generated.

The server computer 16 comprises a computer or other programmable electronic device that includes a non-transitory computer readable medium comprising a database 30 storing clinical activity data 32 indicative of clinical activities of a plurality of medical professionals. The database 30 can also store quality metrics and thresholds for procedures that can be identified from the clinical activity data 32. The database 30 may also comprise multiple databases—for example, the illustrative medical imaging device 12 may generate machine log data as just described that is stored in a machine log database (not shown), and may also generate imaging examination data including images and associated imaging device setting that are stored in a PACS database (not shown).

The database 30 of the server computer 16 can also store a plurality of educational content units 38 for training of medical professionals who operate the device 12. For example, the educational content unit 38 can comprise an animation, a video, and/or a series of images, showing a “best” instance of the procedure, or alternatively an instance of the procedure in which a mistake was made (i.e., to highlight the mistake in the procedure), or a “non-optimal” or “non-recommended usage” where there is a lower quality threshold that the logged procedure fell below. As used herein, the term “suboptimal performance” instance (and variants thereof) refers to an instance of the procedure in which a mistake was made, a non-optimal instance, or a non-recommended usage. In a common implementation, the server computer 16 may be a server computer owned or leased or otherwise under the control of the vendor of the medical device 12, and the clinical activity data 32 (or portions thereof) are uploaded from the database 30 to the vendor's server computer 16 on an occasional basis (e.g., daily). In this case, the analysis of the log data can be performed at the server computer 16 using the copy of the database content stored at the server computer 16. In another example, the educational content units 38 are stored in an external server computer (not shown) owned by an entity other than a vendor of the medical device 12.

The database 30 stores instructions executable by the server computer 16 to perform an educational refreshment method or process 100 implemented by the educational support system 10 for recommending the educational content units 38 for the device 12. In some examples, the method 100 may be performed at least in part by cloud processing (that is, the server computer 16 may be implemented as a cloud computing resource comprising an ad hoc network of server computers).

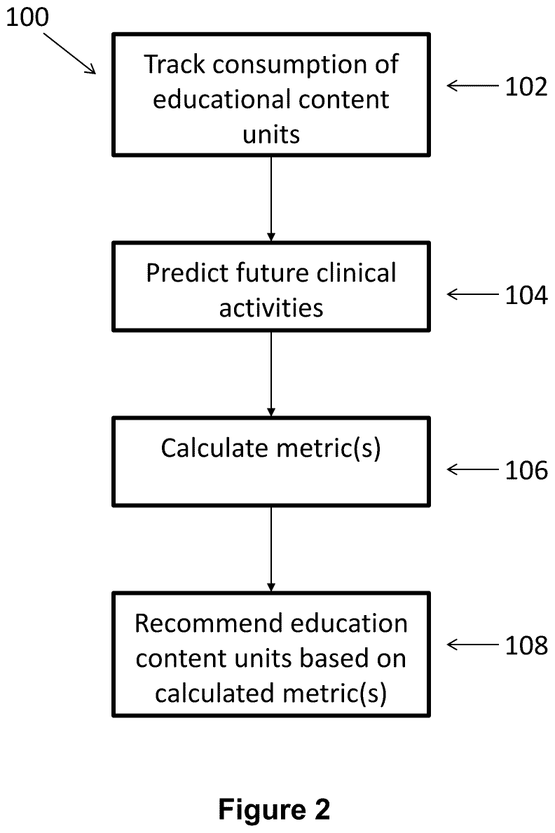

With reference to FIG. 2, and with continuing reference to FIG. 1, an illustrative embodiment of an instance of the method 100 is diagrammatically shown as a flowchart. At an operation 102, consumption of the educational content units 38 by a medical professional are tracked. This can be performed by analyzing which educational content units 38 are viewed or interacted with by of each medical professional.

At an operation 104, future clinical activities to be performed by the medical professional are predicted based on the clinical activity data 32. The future clinical activities can also be predicted based on one or more of a schedule of the medical professional, statistics related to performance of procedures by the medical professional, and data related to the one or more medical devices 12.

At an operation 106, one or more metrics 40, 42 (stored in the database 30) related to the medical professional's knowledge and/or experience for a future time are calculated. The metrics 40, 42 can include (i) a knowledge metric 40 based on the tracked consumption of educational content units 38, an experience metric 42 calculated based on the clinical activity data and the predicted future clinical activities, and so forth. The knowledge metric 40 can be calculated taking into account an erosion of knowledge over a time interval ending at the future time over which the erosion of knowledge of the medical professional occurs. The experience metric 42 can be calculated taking into account an erosion of experience over a time interval ending at the future time over which the erosion of experience of the medical professional occurs. In some examples, the calculated metric(s) 40, 42 relate to the medical professional's knowledge and/or experience relating to a future clinical activity of the future clinical activities predicted to be performed by the medical professional. The calculated metric(s) 40, 42 can be calculated for one or more future times, e.g. two days ahead, one week-ahead, two weeks ahead, three weeks ahead, et cetera. The metrics can also be calculated for various clinical activities, such as those predicted in the operation 104.

At an operation 108, the one or more refreshment educational content units 38 are recommended for each medical professional based on the metric(s) 42 meeting an educational content refreshment criterion. For example, if the calculated metrics indicate a knowledge and/or experience gap, then one or more refreshment educational content units 38 are identified and recommended to address the gap. Advantageously, because the calculated metric(s) 40, 42 take into account erosion of knowledge and experience over time, the identified gaps take such erosion into account. This can be done on a per-medical professional basis, to provide personalized refreshment educational recommendations. Referring back to FIG. 1, the educational content unit(s) 38 recommended in operation 108 may be consumed in various ways. In one illustrative example, the server computer 16 is in communication with an electronic processing device 18, such as a workstation computer located in at the customer (e.g. hospital). The illustrative workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18 or may include two or more display devices. The electronic processor 20 is operatively connected with one or more non-transitory storage media 26. The non-transitory storage media 26 stores instructions executable by the at least one electronic processor 20 to present the educational content unit(s) 38 received from the server computer 16. For example, these instructions may implement a video player to present educational content comprising video content, and/or an image display program to present educational content comprising still images, and/or an animation presentation program to present educational content stored in an animation file format, and/or so forth. The instructions optionally include instructions to generate a graphical user interface (GUI) 28 for display on the display device 24 that coordinates the presentation of the educational content, especially if the educational content unit(s) 38 are multimedia content in the sense that the educational content is provided in multiple formats (e.g., video, still images, and animations). For example, a content-editing UI, a video editing UI, and/or an image editing UI, can be displayed on the GUI 28.

The educational content units 38 can then be stored in the server computer 16 (and their associated metrics 40, 42 and links to associated data of the device 12) and/or at the non-transitory storage medium 26. The educational content generator may optionally also include a matching algorithm (not shown) implemented by the server computer 16 that suggests educational content units to users based on information on the users such as performance ratings. Hence, remedial content units may be suggested to users who do not exceed some performance rating threshold of good practice, or suggests content on avoidance of common mistakes to users who are new to the system or are below the lower threshold and exceed a certain frequency threshold (i.e. that error occurs frequently enough). Each best/worst case simulation is optionally overwritten (or archived) when new data generated in the field represents a closer/further result to the current best/worst values.

In some embodiments, when the educational content units 38 are stored in the database 30, the educational refreshment method 100 can include retrieving the one or more stored refreshment educational content units 38 and presenting the retrieved one or more refreshment educational content units 38 to the medical professional at a time prior to the future time.

In some embodiments, the educational refreshment method 100 can include dividing the recommended educational content unit(s) (38) into one or more segments, and optionally tagging the one or more segments with a quantitative activity content tag.

In another contemplated variant, feedback from the medical professional about the timeliness of the recommendation of educational content units in operation 108 can be sought from the medical professional before, during, or after delivery of the recommended educational content. For example, after delivery of an educational content unit to the medical professional, a brief questionnaire can be presented asking whether the educational content unit was useful, and/or whether the educational content unit was presented in a timely manner. Alternatively, rather than directly asking for this information, the educational content unit may include a content-related quiz presented before commencing presentation of the educational content unit and again after the presentation of the educational content unit. If the medical professional obtains a low score on the quiz presented before the content unit commences but a high score on the follow-up quiz presented after presentation of the content unit, then it may be inferred that the presentation of the educational content unit was timely since it resulted in acquisition of knowledge from the content unit by the medical professional. On the other hand, if the medical professional achieves high scores on both the before and after quizzes, then it can be inferred that the content unit was presented prematurely, since the medical professional already knew much of the substance of the content unit beforehand as evidenced by the high score on the “before” quiz. The feedback on timeliness of the content unit presentation (whether solicited directly or indirectly, e.g. by content-related quizzes) can be used to tune the calculation of the one or more metrics 40, 42 in the operation 106. For example, the tuning could involve adjusting kₐ based on the feedback to tune the rate of knowledge erosion, and/or adjusting eₐ to tune the rate of experience erosion, in the operation 106.

In some embodiments, the educational refreshment method 100 can include tracking, based on the quantitative activity content tag, a time at which the metric(s) 40, 42 will underrun the predetermined threshold, and recommending the educational content unit(s) 38 to the medical professional before the time at which the one or more metrics will underrun the predetermined threshold.

### EXAMPLE

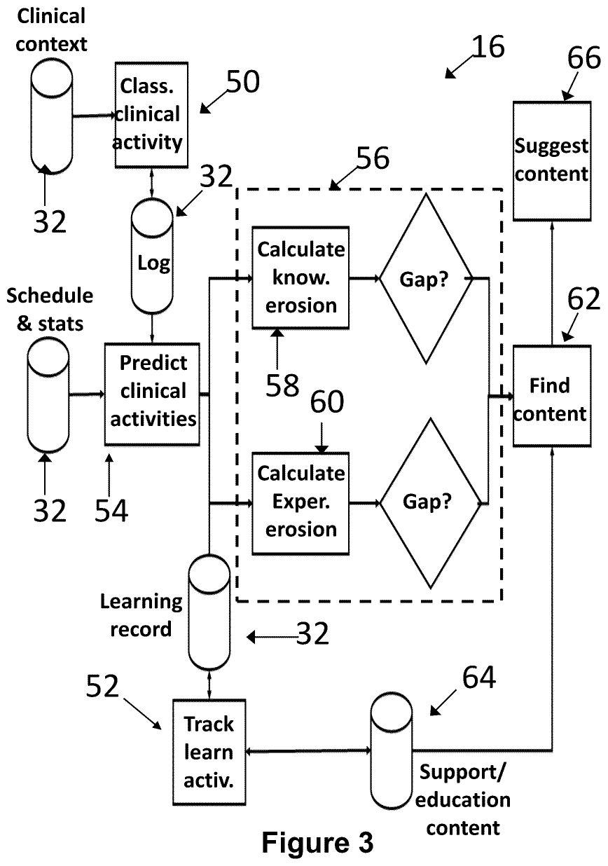

The following provides another example of the educational content monitoring system or apparatus 10. With reference to FIG. 3, and with continuing reference to FIGS. 1 and 2, a clinical activities processor 50 (implemented in the server computer 16) is programmed to track and interpret the user's clinical activities/procedures in the field so that they may be classified within a database of historical activities. This is essential to establish the gaps in the user's experience level for each clinical activity classification.

Each classification is broken down into activity type, patient case type, identified complications during activities and other statistical properties around each type that may have a link to educational or support related content unit 38 (such as medical guidelines or triage checklists). The clinical activities processor 50 is not restricted to a particular type of machine learning method to perform the classification.

The data sources for the clinical and case specific activities include machine log data, hospital information data, patient case data, sensor data about the workflow steps performed

In addition to classifying clinical activities, the system 10 can be generalized to also encompass maintenance activities too, whereby biomed activities can also be classified for the detection of gaps in experience.

A learning status processor 52 (implemented in the server computer 16) is programmed to track the user's learning status (i.e. learning activities) and learning history to maintain a record of learning. This enables establishing the gaps in knowledge of the user at any time, together with their historical clinical activity data. The learning status processor 52 identifies what case-specific content has been taught and when it was taught, along with which content was previously consumed, including online reference materials, webinars, seminars, conferences, application specialist trainings/calls, and so forth.

A predictive analytics processor 54 (implemented in the server computer 16) is programmed to predict the types of clinical activity deviations expected to be conducted in the near future for (i.e., from minutes to weeks). The processor uses the following context information to predict future activities and their expected time of occurrence based on, for example, schedules of the clinical staff and the schedules of the planned procedures, rooms, and devices; ambulatory information about incoming patients to a hospitals; seasonal variations in the context of required clinical activities; staff changes and capacity forecasts with respect to required competencies; and so forth.

A learning re-enforcement processor 56 (implemented in the server computer 16) is programmed to identify and recommend candidates for supporting content units 38 that can be consumed by the user based on their gaps in knowledge, predicted activities and the timeliness to act on the content before they must perform the activity. A prioritized list of case specific learning content based on the frequency and regularity of case-related activities/procedures conducted. Cases that have had the lowest frequency or regularity will be used as a basis to reinforce learning.

In one embodiment of the learning re-enforcement processor 56, consider a user with a given amount of knowledge, k, and experience, e, in a particular clinical activity type, kₐ and eₐ (see below for the attributes of kₐ and eₐ). The system 10 keeps track of a learning record of the user, which registers all of the interactions of the user with respect to learning materials in a learning management system, support materials, guidelines access, webinars, etc. Each of these records contains information about the last time such content was consumed, as well as how relevant the content is with respect to the expected tasks of the user (their role and competencies) and with respect to their hospital's workflows and devices. The knowledge of a user for a given activity kₐ can be defined as a decimal number between 0 and 1, where 0 indicates no knowledge, and 1 indicates maximum knowledge (i.e. expert).

The system 10 also keeps track of the actual clinical activities that the user has performed. It does this by fusing data sources from the hospital information systems and the medical device log data to infer the types of clinical activities (procedures) that were performed, the sub-steps within each activity, the configurations of the devices within each sub-step, the patient case data (anonymized) relating to each activity, outlier information about the activity (e.g., the time taken was longer than average, certain sub-steps were repeated, etc.). The system also registers the last time such a specific clinical activity was performed with those attributes (taking patient case-specific information into account). Similarly, the experience of a user for a given activity eₐ can be defined as a decimal number between 0 and 1, where 0 indicates no experience, and 1 indicates maximum experience (i.e. repeated activity on regular (e.g. daily) basis).

At regular intervals, the system analyses the frequency and regularity (e.g., calculated knowledge erosion metric 58 and calculated experience erosion metric 60) of the user applying each type of clinical activity in the field, as well as the frequency and regularity of the consumption data from the learning system, against some form of threshold. This is included as part of a knowledge erosion model, where the knowledge and experience of a user decays over time if they are not reading new material and not applying their knowledge, respectively. This can be realized with the following model:

$${k}_{a} ( t + δ ) = min ( 0 , max ( {k}_{a} ( t ) - λ * δ + r ( t , t + δ ) , 1 ) )$$

$${e}_{a} ( t + δ ) = min ( 0 , max ( {e}_{a} ( t ) - θ * δ + p ( t , t + δ ) , 1 ) )$$

Where eₐ(t) and kₐ(t) are the knowledge/experience for a given activity at time t, λ/θ are the decay factors of the knowledge/experience, r(t,t+δ) is the normalized number of content read by the user on activity a between time t and t+δ, and p(t,t+δ) is normalized amount activities of activity a performed by the user between time t and t+δ. Both r and p are values between 0 and 1.

From this, it will suggest learning refreshment opportunities (ranked content) based on discovered content that matches the specific clinical activity attributes for which the frequency and regularity is low. In the illustrative embodiment, in an operation 62 a support/education center or other source 64 of educational content units is searched to find supporting content for remedying the identified gap(s), and the content may optionally be ranked on relevance to the future predicted activities of the medical professional. In an operation 66 this content is suggested to the medical professional (and, in some embodiments, is actively pushed or delivered to the medical professional).

At regular intervals, the system 10 will predict the future activities of the user, using available data sources, including the scheduling system of planned activities, the patient records which containing case-specific information that might lead to deviations in clinical activities, referring physician notes, etc.

If the attributes relating to predicted/planned procedure are new to the user or the erosion metrics of their knowledge or experience exceed a threshold, then the system prioritizes discovered content that is consumable within the time remaining before the activity is expected to take place. The discovered content should match the attributes for which the knowledge gaps are most identifiable and may include sections of textual guidelines, diagrams, videos, e-learning courses, simulator sequences.

In emergency care situations, the time available to prepare for novel clinical activities will be small and thus any recommended content will have to fit within such timescales to make it actionable.

In scheduled care situations (e.g., in a Cath lab setting), where the available time to prepare may range from days to weeks, the system can prioritize larger blocks of supporting content to help learn about the procedure as part of their preparation.

In larger time scales, whereby certain clinical procedures are expected to occur within weeks or months, the system can recommend educational courses to the user (assuming that such gaps in knowledge about the procedures are sufficiently large).

The method 100 of recommending content can take any form in order to identify and match the content and to rank the content in terms of its relevancy to the user. The most relevant content should at least be the most urgent content in terms of the planning time window and should match the largest gaps in the knowledge for the procedures that are most probable to take place.

In the cases where newly defined content (such as new guidelines around clinical procedures) supersedes existing content, only the delta between the existing consumed content and the added content will be presented to the user.

In its simplest embodiment, the system 10 can analyze the worker schedule and patient schedule each week If it notices that a particular clinician has a procedure that they have not recently performed and their training was >1 year ago the system can provide tailored refresher course to help the clinician prepare for the procedure.

The illustrative embodiments are directed to providing educational refreshment for clinical staff which provides healthcare professionals with up-to-date training materials, while accounting for erosion of knowledge and experience over time. However, knowledge erosion is not restricted to only clinical staff in a hospital, and the disclosed educational refreshment techniques can also be applied to provide educational refreshment for other types of professionals. For example, an analogous system can be applied to provide educational refreshment for biomedical engineers or to field service engineers (more generally, medical equipment servicing personnel) that must maintain their knowledge on servicing, maintaining, and repairing medical equipment. Adaptation of the illustrative educational refreshment system for providing educational refreshment for medical equipment servicing personnel entails replacing the clinical activity data 32 indicative of clinical activities of medical professionals with servicing activity data indicative of medical equipment servicing activities of medical equipment servicing personnel. The servicing activity data may be obtained, for example, from maintenance logs or prepared maintenance reports for the medical device 12. Similarly, the educational content units 38 for training of medical professionals who operate the device 12 are suitably replaced by educational content units related to training medical equipment servicing personnel on maintenance tasks for maintaining the medical device 12. The calculation of knowledge and experience metrics related to a medical equipment servicing person's knowledge and/or experience for a future time is then performed analogously to the already-described calculation of clinician knowledge and experience metrics 40 and 42, and one or more refreshment educational content units may then be recommended based on the calculated knowledge and/or experience metrics meeting an educational content refreshment criterion.

In application, a medical training and education delivery platform is used to deliver appropriate training based on the medical professional's personal experience. In order to obtain information that is directly relevant to an individual personal experience, the medical training and educational delivery platform includes dedicated connections to data sources that would provide information on the clinical experience of the medical professional. Such connections could include connections to one or more medical devices, such as an imaging system or monitoring device, patient database, and/or scheduling information. The connection to the medical device could provide information, including log files and system parameters or configurations, that would provide insight into how the medical professional interacted with the medical device. The information to the patient database could provide information pertinent to the medical condition for which the patient was to be treated, the treatment the patient received and/or the patient outcome. The connection to the hospital scheduling system could provide information on the type of upcoming procedures and/or examinations that have been scheduled. Each of these connections must be secure between the source and the platform in order to maintain the protection and privacy of the data transmitted. It is further required to ensure that the connections are all aligned within the hospital information technology infrastructure to ensure safe and secure interconnectivity.

The medical training and education delivery platform uses the data collected and the methods described above to determine what additional training to deliver to the individual. In obtaining the information for the medical device, the training and education platform analyzes information in the form of log files to determine the procedure that was conducted and any abnormalities. Such information is compared versus a database of clinical cases of similar clinical procedures to determine issues pertaining to frequency and abnormalities. Further information, such as outcomes data, can provide further clarity on whether changes to the procedure could result in improved outcomes.

In providing relevant training and educational information, the platform determines the most relevant training information based on information about the healthcare professional's experience and upcoming procedures. In this manner, the platform can segment the training and educational information to the specific points of interest or need for the healthcare professional. This is important as healthcare professionals must retain a lot of information in an ever-developing field and simply do not have the time or resources to obtain more information than is needed for the upcoming activities. The training and educational information can be provided as portions of training or instructional videos, as written explanations of procedures, as statistical analysis of how clinical procedures can be delivered more efficiently or efficiently, or some combination thereof. As most clinical procedures include detailed steps and/or interactions with complex medical devices, it is highly advantageous to provide the training and educational content via one or more simulations. In this regard, the medical professional can interact with the platform to demonstrate how the procedure can be improved. For example, in cases in which a medical device is used in the procedure, the training and educational information can be a simulation of the medical device. Information can be provided on how to use the device more efficiently and/or more effectively and the simulation can be used to determine whether the healthcare provider has understood the content and can implement it in practice. The results of the simulation can further be used to determine if additional or different training and educational information can be provided to assist the healthcare provider. In some situations, the simulation may demonstrate proficiency in the intended training and educational content, but highlight different areas of potential improvement. If this additional area of improvement is within the same procedure, the platform can continue the delivery of the content; whereas if the additional area of improvement is in a related area or step, then the platform can provide the additional training and educational content at some future point in time. If the platform determines to deliver the content at a future point in time, it weighs the additional area of improvement as part of the weighting function and the upcoming schedule to determine what training and educational content should be delivered to the healthcare professional.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A training and education delivery platform for a healthcare provider comprising:
one or more dedicated and secure data connections to a medical device, a clinical activity database indicative of clinical activities of a plurality of healthcare providers, and a patient scheduling database; and
a non-transitory computer readable medium including instructions readable and executable by at least one electronic processor to perform an educational refreshment method comprising:
obtaining medical device log data from a medical device via one of the dedicated and secure data connections;
obtaining clinical activity data from the clinical activity database via one of the dedicated and secure data connections;
obtaining upcoming patient schedule data from the patient securing database via one the dedicated and secure data connections;
tracking consumption of educational content units related to one or more medical procedures by a healthcare provider;
predicting future clinical activities to be performed by the healthcare provider based on the medical device log data, the clinical activity data, and the upcoming patient schedule data;
calculating one or more metrics related to the healthcare provider's knowledge and/or experience for a future time, the calculating including (i) calculating an erosion of knowledge metric over a time interval ending at the future time over which the erosion of knowledge of the healthcare provider occurs based on the tracked consumption of educational content units and/or (ii) calculating an erosion of experience metric over the time interval ending at the future time over which the erosion of experience of the healthcare provider occurs based on the clinical activity data and the predicted future clinical activities;
dynamically updating the one or more metrics based on changes to the medical professional's knowledge and/or experience; and
recommending one or more refreshment educational content units based on the one or more metrics meeting an educational content refreshment criterion; and
a display that provides the medical professional the recommended one or more refreshment educational content units.

2. The training and education delivery platform of claim 1, wherein predicting future clinical activities performed by the healthcare provider is further based on statistics related to performance of procedures by the healthcare provider.

3. The training and education delivery platform of claim 1, wherein the refreshment educational content units are divided into one or more segments.

4. The training and education delivery platform of claim 3, wherein the refreshment educational content units include a quantitative activity content tag.

5. The training and education delivery platform of claim 1, wherein the erosion of knowledge metric is calculated according to:
$${k}_{a} ( t + δ ) = min ( 0 , max ( {k}_{a} ( t ) - λ * δ + r ( t , t + δ ) , 1 ) )$$
and the erosion of experience metric is calculated according to:
$${e}_{a} ( t + δ ) = min ( 0 , max ( {e}_{a} ( t ) - θ * δ + p ( t , t + δ ) , 1 ) )$$
where kₐ(t) and eₐ(t) are the knowledge and experience, respectively, for a given activity at time t, λ and θ are decay factors of the knowledge and experience, respectively, r(t,t+δ) is a normalized number of content read by the user on activity a between time t and t+δ, and p(t,t+δ) is a normalized amount of activity a performed by the user between time t and t+δ.

6. The training and education delivery platform of claim 5, wherein the erosion of knowledge metric is calculated according to:
$${k}_{a} ( t + δ ) = min ( 0 , max ( {k}_{a} ( t ) - λ * δ + r ( t , t + δ ) , 1 ) )$$
where kₐ(t) is the knowledge for a given activity at time t, λ is a decay factor of the knowledge, and r(t,t+δ) is a normalized number of content read by the user on activity a between time t and t+δ.

7. The training and education delivery platform of claim 5, wherein the erosion of experience metric is calculated according to:
$${e}_{a} ( t + δ ) = min ( 0 , max ( {e}_{a} ( t ) - θ * δ + p ( t , t + δ ) , 1 ) )$$
where eₐ(t) is the experience for a given activity at time t, θ is a decay factor of the experience, and p(t,t+δ) is a normalized amount of activity a performed by the user between time t and t+δ.

8. The training and educational delivery platform of claim 1, wherein a portion of the educational content delivered is a simulation of a clinical procedure.

9. The training and educational delivery platform of claim 8, wherein the simulation includes simulating use of a medical device used in the clinical procedure.

10. The training and educational delivery platform of claim 8, wherein the simulation records inputs from the healthcare provider and generates one or more outputs in response to the inputs from the healthcare provider.

11. The training and educational delivery platform of claim 8, wherein the outputs in response to the inputs from the healthcare provider modify the simulation.

12. The training and educational delivery platform of claim 8, wherein the simulation captures a set of results that are used as inputs to the calculation of the one or more metrics related to the healthcare provider's knowledge and/or experience.
